(19) United States
(12) Patent Application Publication
(10) Pub. No.: US 2019/0046513 A1
(43) Pub. Date: Feb. 14, 2019

(54) COMBINATION THERAPIES OF HDAC INHIBITORS AND TUBULIN INHIBITORS

(71) Applicant: HUYA Bioscience International, LLC, San Diego, CA (US)

(72) Inventors: Reid P. Bissonnette, Carlsbad, CA (US); Alain Rolland, San Diego, CA (US); Robert Goodenow, San Clemente, CA (US); Mireille Gillings, Winchester CDP, NV (US)

(21) Appl. No.: 15/676,905

(22) Filed: Aug. 14, 2017

Related U.S. Application Data
(60) Provisional application No. 62/543,770, filed on Aug. 10, 2017.

(51) Int. Cl. A61K 31/4406; A61K 31/357; A61P 35/00; A61P 35/04; A61K 45/06; A61K 31/395

(52) U.S. Cl. CPC A61K 31/4406; A61K 31/357; A61K 31/395; A61P 35/04; A61K 45/06; A61P 35/00

(57) ABSTRACT

Provided herein are combinations that include a histone deacetylase inhibitor and tubulin inhibitor such as eribulin or a pharmaceutically acceptable salt thereof that are useful for treating cancer, including reducing cancer metastasis.

### CROSS-REFERENCE TO RELATED APPLICATIONS

The present invention claims priority to U.S. Provisional Application No. 62/543,770, filed Aug. 10, 2017, entitled “Combination Therapies of HDAC Inhibitors and Tubulin Inhibitors,” which is hereby incorporated in its entirety including all tables, figures, and claims.

### FIELD OF THE DISCLOSURE

The present disclosure relates to combinations of an HDAC inhibitor and a tubulin inhibitor, such as eribulin or a pharmaceutically acceptable salt thereof and the use of such combinations to treat cancer.

### BACKGROUND OF THE DISCLOSURE

Cancer is a significant cause of morbidity and mortality worldwide. While the standards of care for many different cancer types have greatly improved over the years, current standards of care still fail to meet the need for effective therapies to improve treatment of cancer. Breast cancer is the second leading cause of cancer death among women and the median survival of metastatic breast cancer has remained, for many decades, at two to three years after diagnosis. Most deaths due to cancers, including metastatic breast cancer, are the result of dissemination of cancer cells resulting in the formation of secondary tumors (metastasis) in distant organs (Saxena, M. and Christofori, G., (2013) Molecular Oncology 7(2):283-296). Metastasis is indicated to account for more than 90% of cancer-related mortality and morbidity (Chaffer, C. L., and Weinberg, R. A., (2011) Science 331:1559-1564). Most existing cancer drugs only inhibit cancer proliferation. Thus, prevention and/or reducing cancer metastasis is an urgent therapeutic need.

### SUMMARY OF THE INVENTION

Provided herein are combinations that include a histone deacetylase inhibitor and tubulin inhibitor, such as eribulin. The combinations include histone deacetylase inhibitors (HDACi) of formula I and tubulin inhibitors, such as eribulin. In certain instances, the eribulin or a pharmaceutically acceptable salt thereof is, e.g., eribulin mesylate.

When used herein the term “tubulin inhibitor” means an agent that interferes with the tubulin system resulting in inhibition of cell division. Examples of tubulin inhibitors useful for the methods and combinations disclosed herein include without limitation, colchicine analogues, paclitaxel and agents that bind the paclitaxel binding domain and vinca alkaloids and agents that bind the vinca alkaloid binding domain. Tublin inhibitors useful for the invention include, without limitation, paclitaxel, epothilone, docetaxel, discodermolide, colchicine, combrestatin, 2-methoxyestradiol, methoxy benzenesulfonamides (E7010), vinblastine, vincristine, vinorelbine, vinfluine, dolastatins, halichondrins, herniasterlins and cryptophysin 52. Dosages of such tubulin inhibitors can be determined using product inserts of the individual drugs or published literature reporting clinical trials using the tubulin inhibitors.

When used herein the term “eribulin” means eribulin or a pharmaceutically acceptable salt thereof, e.g., eribulin mesylate.

When used herein the term “histone deacetylase inhibitor(s) (HDACi)” means a class of compounds that interfere with the function of histone deacetylase. Exemplary HDACi of the invention include, without limitation, vorinostat, romidepsin, HBI-8000 (chidamide), panobinostat, valproic acid, mocetinostat, abexinostat, entinostat, SB939, reminostat, givinostat, quisinostat, kevetrin, CUDC-101, AR-42, CHR-2845, CHR-3996, 4SC-202, CG200745, ACY-1215, ME-344, sulforaphane, and belinostat. Other HDACi comprise compounds as described in Formula I, which includes HBI-8000. HBI-8000 is a benzamide that inhibits class I HDAC1, HDAC2, HDAC3, and Class II, and HDAC10. HBI-8000 is on the market in China for the treatment of relapse or refractory peripheral T-cell lymphoma.

In a first aspect is a combination that includes a therapeutically effective amount of a tubulin inhibitor, such as eribulin or a pharmaceutically acceptable salt thereof, e.g., eribulin mesylate and a therapeutically effective amount of an HDACi, such as a compound of formula I:

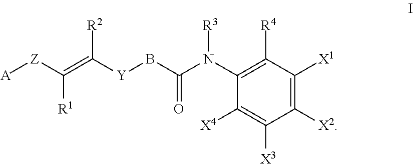

A is phenyl or a heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group. B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl. Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively. Z is a bond or C₁-C₄ alkylene, —O—, —S—, —NH—, —CO—, —CS—, —SO—, or —SO₂—. R¹ and R² are independently hydrogen or C₁-C₄ alkyl. R³ is hydrogen or C₁-C₄ alkyl. R⁴ is hydrogen or —NH₂. One of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen, provided, however, that when R⁴ is hydrogen, one of X¹, X², X³, or X⁴ is —NH₂, an aminoalkyl group or an alkylamino group. In some aspects of the disclosure z is a bond. In other aspects of the invention R³ is hydrogen. In still other aspects A is pyridine. In some aspects, X² is halogen such as fluro. In some aspects R¹ and R² are independently hydrogen. In other aspects R³ is hydrogen. In still other aspects R⁴ is —NH₂. In still other aspects Y is —C(O)NH—CH₂.

In one embodiment, the HDACi inhibitor is N-(2-amino-4-fluorophenyl)-4-[[[(2E)-1-oxo-3-(3-pyridinyl)-2-propen-1-yl]amino]methyl]benzamide, referred to herein as HBI-8000, or chidamide.

In some embodiments, the HDACi has the following structure:

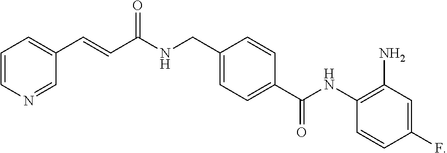 [SMILES: Nc1cc(F)ccc1NC(=O)c1ccc(CNC(=O)/C=C/c2cccnc2)cc1]

In some embodiments a compound of formula I is present at an amount of greater than about 5 mg, or about 5 mg to about 50 mg.

In some embodiments, eribulin or a pharmaceutically acceptable salt thereof is present at amount of about 3 mg/ml or less, or about 0.4 mg/ml to about 1 mg/ml, or about 0.5 mg/ml.

In one aspect the combination comprises eribulin or pharmaceutically acceptable salt thereof.

In some embodiments, the combination is suitable for administration to a cancer patient. In some aspects, the combination comprises a pharmaceutically acceptable excipient.

In an additional aspect the disclosure herein includes kits for (i) use in reducing metastasis of a primary tumor; (ii) preventing or delaying recurrence of the cancer; (iii) extending disease- or tumor free survival time; (iv) increasing overall survival time; (v) reducing the frequency of treatment; (vi) relieving one or more symptoms of the cancer, and (vii) reducing tumor burden. The kits include an HDACi and tubulin inhibitor, such as eribulin or pharmaceutically acceptable salt thereof. In some aspects of the kit, the HDACi and tubulin inhibitor such as eribulin or pharmaceutically acceptable salt thereof comprise separate formulations. In some aspects the tubulin inhibitor, such as eribulin and HDACi are in different containers. The kits can include instructions for use and/or reagents and medical devices for administration.

In another aspect, the disclosure herein provides methods for treating a subject with a primary cancer with a combination of an HDACi and tubulin inhibitor, such as eribulin or a pharmaceutically acceptable salt thereof whereby the treatment results in one or more of the following: (i) reduces or slows tumor metastasis; (ii) prevents or delays recurrence of the cancer; (iii) extends disease- or tumor free survival time; (iv) increases overall survival time; (v) reduces the frequency of treatment; (vi) relieves one or more symptoms of the cancer or combinations of the aforementioned, and (vii) reduces tumor burden.

In some embodiments, the primary tumor is treated by one or more of radiation, surgery, chemotherapy, immunotherapy, targeted therapy, hormone therapy, stem cell transplant, cryotherapy, laser therapy, and precision medicine. In some aspects of this embodiment, the combination is administered prior, concurrently, subsequently, or combinations of prior, concurrently and subsequently to treatment of the primary tumor. In some aspects, the HDACi is used alone to prime the tumor for a period of time before treatment using the combination. The period for priming can be 1 day, 2 days, 3 days, 4 days, 5 days, 6 days, one week, greater than one week, two weeks, greater than two weeks, three weeks, or greater than three weeks. In some aspects of the priming, the HDACi is administered bi-weekly or tri-weekly for a period of time before treatment using the combination begins.

In some embodiments of the methods disclosed herein the metastasis that is reduced is metastasis of one or more of the adrenal gland, brain and/or spinal cord, bone, lung, liver and/or pleura, gastrointestinal tract, peritoneum, muscle, lymph nodes and skin.

In some embodiments of the methods disclosed herein, the primary tumor or secondary tumor of the subject being treated with the combination is a cancer of the breast, lung, bladder, skin, intestine, colon, kidney, ovary, pancreas, prostate, stomach, thyroid, head and neck, gastroesophageal tract, connective or other nonepithelial tissue, lymphatic cells and uterus. In some aspects of this method, the cancer is triple negative breast cancer.

In some embodiments of the methods, the methods further comprise treatment of the subject with an E-selectin inhibitor, or plerixafor, or a combination of an E-selectin inhibitor and plerixafor. In some aspects of this method the E-selectin inhibitor and/or plerixafor is given prior, concurrently, or subsequently, or combinations of prior, concurrently or subsequently, to the HDACi and tubulin inhibitor, such as eribulin, combination. In still further aspects of this embodiment, treatment further comprises treating the subject with an αv integrin inhibitor, or an antibody from the group comprising etaracizumab, intetumumab, or abituzumab or a combination of an αv integrin inhibitor and an antibody from the group comprising etaracizumab, etaracizumab, intetumumab, or abituzumab. In other aspects of this embodiment, treatment further comprises treating the subject with a matrix metalloproteinase inhibitor, wherein said matrix metalloproteinase inhibitor is given prior, concurrently, or subsequently, or combinations prior, concurrently or subsequently, to the HDACi and tubulin inhibitor combination.

The combination/methods disclosed herein can result in synergistic effects in which the effects of the combination are greater than the sum of the effects of the drugs when administered alone. Combination methods that have additive effects are also beneficial for the methods disclosed herein.

### BRIEF DESCRIPTION OF THE DRAWINGS

FIG. 1 shows the individual tumor growth curves for all study groups in Study Group A. Each line represents tumor growth in an individual animal.

FIG. 2 depicts the effect of each treatment on the number of metastatic nodules counted for each treatment group in Study Group B.

FIG. 3 shows the number of metastatic nodules in each group (example 2).

FIG. 4 shows tumor volume distribution in each group (example 2)

FIG. 5 shows a linear regression correlation analysis for all groups vs. CDH1 gene expression against metastatic foci count

FIG. 6 shows a linear regression correlation analysis for all groups vs. OCLN gene expression against metastatic foci count.

FIG. 7 shows a linear regression correlation analysis for all groups vs. CLDN1 gene expression against metastatic foci count.

FIG. 8 shows a linear regression correlation analysis for all groups vs. CCL5 gene expression against metastatic foci count.

FIG. 9 shows a linear regression correlation analysis for all groups vs. HMGA2 gene expression against metastatic foci count.

### DETAILED DESCRIPTION OF THE EMBODIMENTS

All patents, applications, published applications and other publications cited herein are incorporated by reference in their entirety. Unless defined otherwise, all technical and scientific terms used herein have the same meaning as commonly understood by those of ordinary skill in the art to which the invention belongs. The chemical structures and formulae set forth herein are constructed according to the standard rules of chemical valency known in the chemical arts. Should a discrepancy exist between a depicted structure and a name given for that structure, the depicted structure is to be accorded more weight. Where the stereochemistry of a structure or a portion of a structure is not indicated in a depicted structure or a portion of the depicted structure, the depicted structure is to be interpreted as encompassing all of its possible stereoisomers.

Any methods, devices and materials similar or equivalent to those described herein can be used in the practice of this invention. The following definitions are provided to facilitate understanding of certain terms used frequently herein and are not meant to limit the scope of the present disclosure. In the event that there is a plurality of definitions for a term herein, those in this section prevail unless stated otherwise. Headings used herein are for organizational purposes only and in no way limit the invention described herein.

The term “effective amount” refers to the amount of a therapy (e.g., a combination provided herein or another active agent such as an anti-cancer agent described herein) which is sufficient to accomplish a stated purpose or otherwise achieve the effect for which it is administered. An effective amount can be sufficient to reduce and/or ameliorate the progression, development, recurrence, severity and/or duration of a given disease, disorder or condition and/or a symptom related thereto. An effective amount can be a “therapeutically effective amount” which refers to an amount sufficient to provide a therapeutic benefit such as, for example, the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy. A therapeutically effective amount of a composition described herein can enhance the therapeutic efficacy of another therapeutic agent.

The term “regimen” refers to a protocol for dosing and timing the administration of one or more therapies (e.g., combinations described herein or another active agent such as an anti-cancer agent described herein) for treating a disease, disorder, or condition described herein. A regimen can include periods of active administration and periods of rest as known in the art. Active administration periods include administration of combinations and compositions described herein and the duration of time of efficacy of such combinations and compositions. Rest periods of regimens described herein include a period of time in which no compound is actively administered, and in certain instances, includes time periods where the efficacy of such compounds can be minimal. Combination of active administration and rest in regimens described herein can increase the efficacy and/or duration of administration of the combinations and compositions described herein.

The terms “therapies” and “therapy” refer to any protocol(s), method(s), and/or agent(s) that can be used in the prevention, treatment, management, and/or amelioration of a disease, disorder, or condition or one or more symptoms thereof. In certain instances the term refers to active agents such as an anti-cancer agent described herein. The terms “therapy” and “therapy” can refer to anti-viral therapy, anti-bacterial therapy, anti-fungal therapy, anti-cancer therapy, biological therapy, supportive therapy, and/or other therapies useful in treatment, management, prevention, or amelioration of a disease, disorder, or condition or one or more symptoms thereof known to one skilled in the art, for example, a medical professional such as a physician.

The term “patient” or “subject” refers to a mammal, such as a human, bovine, rat, mouse, dog, monkey, ape, goat, sheep, cow, or deer. Generally a patient as described herein is human.

The terms “inhibition”, “inhibit”, “inhibiting” refer to a reduction in the activity, binding, or expression of a polypeptide or reduction or amelioration of a disease, disorder, or condition or a symptom thereof. Inhibiting as used here can include partially or totally blocking stimulation, decreasing, preventing, or delaying activation or binding, or inactivating, desensitizing, or down-regulating protein or enzyme activity or binding.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “administering” refers to the act of delivering a combination or composition described herein into a subject by such routes as oral, mucosal, topical, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. Administration generally occurs after the onset of the disease, disorder, or condition, or its symptoms but, in certain instances, can occur before the onset of the disease, disorder, or condition, or its symptoms (e.g., administration for patients prone to such a disease, disorder, or condition).

The term “coadministration” refers to administration of two or more agents (e.g., a combination described herein and another active agent such as an anti-cancer agent described herein). The timing of coadministration depends in part of the combination and compositions administered and can include administration at the same time, just prior to, or just after the administration of one or more additional therapies, for example cancer therapies such as chemotherapy, hormonal therapy, radiotherapy, or immunotherapy. The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compounds described herein can be used in combination with one another, with other active agents known to be useful in treating cancer.

The term “anti-cancer agent” is used in accordance with its plain ordinary meaning and refers to a composition having anti-neoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

The terms “halo,” “halogen,” and “halide” refer to —F, —Cl, —Br, and —I.

The term “alkyl” by itself or as part of another substituent refers to, unless otherwise stated, a straight (e.g., unbranched) or branched carbon chain (or carbon), or combination thereof, having no unsaturation and can include mono-, di- and multivalent radicals. An alkyl as defined herein can be designated by its number of carbon atoms (e.g., C₁-C₁₀ means one to ten carbons). Alkyls herein can include C₁-C₁₀, C₁-C₈, C₁-C₆, and C₁-C₄ lengths. A “perfluoroalkyl” refers to an alkyl in which all of the hydrogens in the alkyl chain are replaced with fluoro.

The term “alkoxy” refers to an alkyl group (e.g., C₁-C₁₀, C₁-C₈, C₁-C₆, and C₁-C₄ alkyl) attached to the remainder of the molecule via an oxygen linker (—O—). Exemplary alkoxy groups include groups having the formula —OR, where R is branched or linear alkyl. A “perfluoroalkoxyl” moiety refers to an alkoxy in which all of the hydrogens in the alkyl chain are replaced with fluoro.

The term “aminoalkyl” refers to an alkyl group (e.g., C₁-C₁₀, C₁-C₈, C₁-C₆, and C₁-C₄ alkyl) in which one or more hydrogen atoms are replaced with an amino group.

The term “alkylamino” refers to an alkyl group (e.g., C₁-C₁₀, C₁-C₈, C₁-C₆, and C₁-C₄ alkyl) attached to the remainder of the molecule via a nitrogen linker (—NR—). Exemplary alkylamino groups include N-methylamino, N-ethylamino, N-isopropylamino, and the like.

The term “acyl” refers to a moiety having the formula, —C(O)R, where R is a substituted or unsubstituted alkyl, haloalkyl, or amino group. The term “acylamino” refers to an acyl moiety having an attached amino group and includes, for example, such moieties as acetylamino, propionylamino, butyrylamino, isobuytrylamino, and others.

The term “alkythio” refers to an alkyl group (e.g., C₁-C₁₀, C₁-C₈, C₁-C₆, and C₁-C₄ alkyl) attached to the remainder of the molecule via a sulfur linker (—S—). Exemplary alkylthio groups include methylthio, ethylthio, propylthio, and others.

The term “heterocycle” or “heterocyclyl” refers to a stable 3- to 15-membered monocyclic group that is saturated or unsaturated and contains one or more heteroatoms (e.g., N, O, or S). Exemplary heterocycles include, but are not limited to morpholinyl, piperidinyl, piperazinyl, pyranyl, pyrrolidinyl, pyrrolinyl, imidazolidinyl, imidazolinyl, oxetanyl, azetidinyl, and others.

### Compositions

Provided herein are combinations (e.g., combination therapies and compositions) useful for treating a variety of diseases, disorders, and symptoms thereof, including for example, cancer. The combinations described herein include tubulin inhibitors, such as eribulin or pharmaceutically acceptable salt thereof, and an HDACi inhibitor, such as benzamide HDACi of formula I as described herein. In one aspect is a combination that includes a therapeutically effective amount of a eribulin or pharmaceutically acceptable salt thereof and a therapeutically effective amount of a compound of formula I:

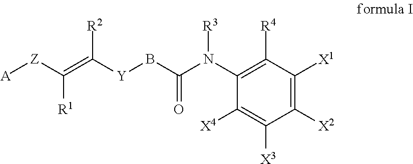

where:

A is a phenyl or heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group;

B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl;

Y is a moiety comprising —CO— which is linear and in which the distances between the centroid of ring B (W1), the centroid of ring A (W2) and an oxygen atom as a hydrogen bond acceptor in the moiety Y (W3) are: W1-W2=about 6.0 Å, W1-W3=about 3.0 Å to about 6.0 Å, and W2-W3=about 4.0 Å to about 8.0 Å, respectively;

Z is a bond or C₁-C₄ alkylene, —O—, —S—, —NH—, —CO—, —CS—, —SO—, or —SO₂—;

R¹ and R² are independently hydrogen or C₁-C₄ alkyl;

R³ is hydrogen or C₁-C₄ alkyl;

R⁴ is hydrogen or —NH₂; and

one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen,

provided, however, that when R⁴ is hydrogen, one of X¹, X², X³, or X⁴ is —NH₂, an aminoalkyl group or an alkylamino group.

In certain instances A is phenyl or phenyl optionally substituted with halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, or a heterocyclic group. A can be a heterocyclic group (e.g., a 5 to 10-membered heterocyclic group) containing a —N—, —S—, or —O— moiety. In certain instances A is a 5 to 10-membered N-heterocyclic moiety having 1, 2, 3, 4, or more nitrogen heteroatoms, such as for example, pyrrolidinyl, pyrrolinyl, imidazolidinyl, imdazolyl, pyrazolidinyl, pyrazolyl, oxazolidinyl, oxazolyl, thiazolidinyl, thiazolyl, piperidinyl, pyridinyl, piperizinyl, diazinyl, tetrazolyl, triazinyl, tetrazinyl, azepinyl, diazepinyl, azocanyl, or azocinyl. A can be a saturated or unsaturated 5 to 10 membered N-heterocyclic moiety. In certain instances A is a 6-membered N-heterocyclic moiety, such as for example, pyridine.

In certain embodiments, B is phenyl. B can be phenyl optionally substituted with a small moiety such as, for example, halogen, —OH, —NH₂, —NO₂, —CN, —COOH, or C₁-C₄ alkyl. In some embodiments B is phenyl substituted with halogen. In other embodiments, B is substituted with an electron donating group (EDG). In still other embodiments, B is phenyl substituted with an electron withdrawing group (EWG). In yet another embodiment, B is phenyl substituted with C₁-C₄ alkyl. B can be methyl-, ethyl-, or propyl-substituted phenyl. B can be methoxy-, ethoxy-, or propoxy-substituted phenyl.

In certain instances Y is —C(O)NH—CH₂—. In certain embodiments, Z is a bond. Z can be a methylene, ethylene, or propylene moiety. In some embodiments, Z is —O—, —S—, —NH—, —CO—, —CS—, —SO—, or —SO₂—.

R¹ and R² are in certain instances both hydrogen. R¹ and R² can both be C₁-C₄ alkyl, for example, R¹ and R² can both be methyl, ethyl, or propyl. In certain instances if one of R¹ or R² is hydrogen the other is C₁-C₄ alkyl (e.g., methyl). R³ can be hydrogen. In other embodiments, R³ is C₁-C₄ alkyl (e.g., methyl or ethyl).

R⁴ can be —NH₂. In certain instances R⁴ is —NH₂ where one of X¹, X², X³, or X⁴ is halogen. When R⁴ is —NH₂, X² or X³ can be halogen. In one embodiment R⁴ is —NH₂ and X² is halogen. In such instances X² can be —F.

In another embodiment, R¹, R², and R³ are hydrogen where Z is a bond, R⁴ is —NH₂ and Y is —C(O)NH—CH₂—. In such embodiments, A can be a heterocyclic moiety as described above and B can be phenyl. X¹, X², X³, or X⁴ can be halogen (e.g., —F) or —NH₂.

The compound of formula I can be a compound as substantially described by U.S. Pat. Nos. 7,244,751 and 7,550,490 both of which are incorporated herein in their entireties for all purposes. In one embodiment the compound of formula I is N-(2-amino-4-fluorophenyl)-4-[[[(2E)-1-oxo-3-(3-pyridinyl)-2-propen-1-yl]amino]methyl]benzamide. In another embodiment the compound of formula I has the structure as set forth below in formula Ia:

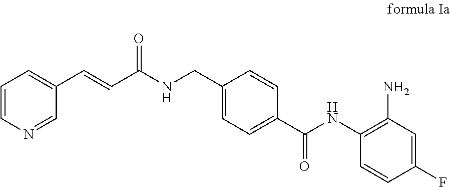 [SMILES: Nc1cc(F)ccc1NC(=O)c1ccc(CNC(=O)/C=C/c2cccnc2)cc1]

Compounds of formula I as described herein include pharmaceutically acceptable salts, pharmaceutically acceptable stereoisomers, prodrugs, enantiomers, diastereomers, hydrates, co-crystals, and polymorphs thereof.

In certain instances, the combination includes a compound of formula I (e.g., Ia) present at an amount of greater than about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount greater than about: 1 mg, 2 mg, 3 mg, 4 mg, 5 mg, 6 mg, 7 mg, 8 mg, 9 mg, or 10 mg. In certain instances the compound of formula I is present in an amount greater than about 5 mg or about 10 mg. The combination can include a compound of formula I present at an amount greater than about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound present in an amount of at least about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount of at least about: 1 mg, 2 mg, 3 mg, 4 mg, 5 mg, 6 mg, 7 mg, 8 mg, 9 mg, or 10 mg. In certain instances the compound of formula I is present in an amount of at least about 5 mg or about 10 mg. The combination can include a compound of formula I present at an amount of at least about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

The combination can include a compound of formula I present in an amount of about: 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. The combination can include a compound of formula I present at an amount of about: 1 mg, 2 mg, 3 mg, 4 mg, 5 mg, 6 mg, 7 mg, 8 mg, 9 mg, or 10 mg. In certain instances the compound of formula I is present in an amount of about 5 mg or about 10 mg. The combination can include a compound of formula I present at an amount of about: 1 mg to about 10 mg, 1 mg to about 25 mg, 1 mg to about 50 mg, 5 mg to about 10 mg, 5 mg to about 25 mg, 5 mg to about 50 mg, 10 mg to about 25 mg, 10 mg to about 50 mg, 50 mg to about 100 mg, or 100 mg to about 200 mg.

A compound of formula I can be present in the combinations described herein relative to the weight of the patient (e.g., mg/kg). In some instances, the compound of formula I is present in an amount equivalent to about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg. In other instances the compound of formula I is present in an amount equivalent to about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg.

The combination also includes eribulin, also known as E7389, ER-086526, NSC-707389 and B1939 which is a synthetic analogue of a polyether macrolide known as halichondrin B. Halichondrin B is isolated from the Japanese sea sponge Halichondria okadai which contain a number of bioactive compounds named halichondrins. The mesylate salt of eribulin with the brand name Halaven® is approved in the U.S. for treating metastatic breast cancer in patients that have had at least two prior chemotherapy regimens for metastasis, including anthracycline and taxane. The mesylate salt of eribulin is also approved in the U.S. for treatment of liposarcoma in patients who have received prior chemotherapy that contained an anthracycline drug. Eribulin is thought to exert its pharmacologic effects by binding to the plus ends of microtubules and suppressing microtubule growth, without affecting microtubule shortening, and by inducing the formation of nonproductive tubulin aggregates.

The chemical name for eribulin mesylate is 11,15:18,21:24,28 Triepoxy-7,9-ethano-12,15-methano-9H, 15H-furo[3,2-i]furo[2′,3′: 5,6]pyrano[4,3b][1,4]dioxacyclopentacosin-5(4H)-one,2-[(2S)-3-amino-2-hydroxypropyl]hexacosahydro-3 methoxy-26-methyl-20,27-bis(methylene)-, (2R,3R,3aS,7R,8aS,9S,10aR,11S,12R,13aR,13bS,15S,18S,21 S,24S,26R,28R,29aS)-, methanesulfonate (salt) and has the structure shown in formula II

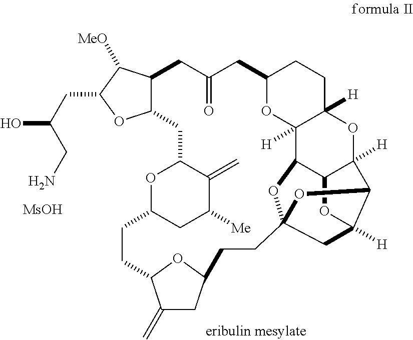

Methods for the synthesis of eribulin (and pharmaceutically acceptable salts thereof, such as eribulin mesylate) are described, for example, in U.S. Pat. No. 6,214,865; U.S. Pat. No. 7,982,060; U.S. Pat. No. 8,350,067; and U.S. Pat. No. 8,093,410, each of which is incorporated herein by reference.

Eribulin or a pharmaceutically acceptable salt thereof can be present in an amount as a measure with regards to the weight of the patient in need thereof. For example, eribulin or a pharmaceutically acceptable salt thereof can be present in an amount of about: 0.1 mg/kg to about 2.0 mg/kg, or about 0.1 mg/kg to about 1 mg/kg. Eribulin or a pharmaceutically acceptable salt thereof can be present in an amount of about: 0.5 mg/kg to about 2.0 mg/kg, or about 0.5 mg/kg to about 1 mg/kg. Eribulin or a pharmaceutically acceptable salt thereof can be present in an amount of about 0.5 mg/kg to about 1.7 mg/kg or about 0.1 mg/kg to about 1.7 mg/kg. Eribulin or a pharmaceutically acceptable salt thereof can be present in an amount of about 0.5 mg/kg to about 1.5 mg/kg or about 0.1 mg/kg to about 1.5 mg/kg.

In still other embodiments, eribulin or a pharmaceutically acceptable salt thereof can be present at an amount of about 0.05-1 mg/kg or about 0.25-1 mg/kg or about 0.25 mg/kg or about 0.5 mg/kg, or 1 mg/kg, or about 0.05 mg/kg, or about 1.3-4.0 mg/kg or about 1.7 mg/kg, or about 1.3 mg/kg, or about 4.0 mg/kg, or 0.375-1.5 mg/kg, or 0.1 mg/kg.

The eribulin or pharmaceutically acceptable salt thereof (e.g., eribulin mesylate) can be administered by intravenous infusion, for example, for about 1 to about 20 minutes, or for about 2 to about 5 minutes. Further, the eribulin or pharmaceutically acceptable salt thereof (e.g., eribulin mesylate) can be administered in an amount in the range of about 0.1 mg/m² to about 20 mg/m², or about 1.4 mg/m² or 1.1 mg/m². In addition, the eribulin or pharmaceutically acceptable salt thereof (e.g., eribulin mesylate) can be administered once on each of days 1 and 8 of a 21-day cycle.

Treatment according to the methods of the disclosure can result in one or more of the following: (i) reduce the number of cancer cells; (ii) reduce tumor volume; (iii) increase tumor regression rate; (iv) reduce or slow cancer cell infiltration into peripheral organs; (v) reduce or slow tumor metastasis; (vi) reduce or inhibit tumor growth; (vii) prevent or delay occurrence and/or recurrence of the cancer and/or extend disease- or tumor-free survival time; (viii) increase overall survival time; (ix) reduce the frequency of treatment; (x) reduction in tumor burden and (xi) relieve one or more of symptoms associated with the cancer.

In certain instances the therapeutically effective amount of the tubulin inhibitor such as eribulin or a pharmaceutically acceptable salt thereof is determined as an amount provided in a package insert provided with the tubulin inhibitor, such as eribulin or a pharmaceutically acceptable salt thereof. The term package insert refers to instructions customarily included in commercial packages of medicaments approved by the FDA or a similar regulatory agency of a country other than the USA, which contains information about, for example, the usage, dosage, administration, contraindications, and/or warnings concerning the use of such medicaments.

HDACi such as compounds of formula I, as described herein, can be provided in amounts that are synergistic with the amount of eribulin or a pharmaceutically acceptable salt thereof. The term synergistic refers to a combination described herein (e.g., HBI-8000 and eribulin or a pharmaceutically acceptable salt thereof—including coadministration with another active agent such as an anti-cancer agent described herein) or a combination of regimens such as those described herein that is more effective than the additive effects of each individual therapy or regimen.

A synergistic effect of a combination described herein can permit the use of lower dosages of one or more of the components of the combination (e.g., a compound of formula I or of eribulin or a pharmaceutically acceptable salt thereof). A synergistic effect can permit less frequent administration of at least one of the administered therapies (e.g., a compound of formula I of eribulin or a pharmaceutically acceptable salt thereof) to a subject with a disease, disorder, or condition described herein. Such lower dosages and reduced frequency of administration can reduce the toxicity associated with the administration of at least one of the therapies (e.g., a compound of formula I of eribulin or a pharmaceutically acceptable salt thereof) to a subject without reducing the efficacy of the treatment. A synergistic effect as described herein avoid or reduce adverse or unwanted side effects associated with the use of any therapy.

Pharmaceutical Compositions

Combinations described herein can be provided as a pharmaceutical composition suitable for administration via any route to a patient described herein including but not limited to: oral, mucosal (e.g., nasal, inhalation, pulmonary, sublingual, vaginal, buccal, or rectal), parenteral (e.g., subcutaneous, intravenous, bolus injection, intramuscular, or intra-arterial), topical (e.g., eye drops or other ophthalmic preparations), transdermal or transcutaneous administration to a patient.

Exemplary of dosage forms include: tablets; caplets; capsules (e.g., gelatin capsules); cachets; lozenges; suppositories; powders; gels; liquid dosage forms suitable for parenteral administration to a patient; and sterile solids (e.g., crystalline or amorphous solids) that can be reconstituted to provide liquid dosage forms suitable for parenteral administration to a patient.

Pharmaceutical compositions and dosage forms described herein typically include one or more excipients. Suitable excipients are well known to those skilled in the art of pharmacy. Whether a particular excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a particular formulation.

Pharmaceutical compositions described herein can in certain instances include additional active agents other than those in the combinations described herein (e.g., an anti-cancer agent such as those described herein) in an amount provided herein.

In one embodiment, the compound of formula I is provided in an oral dosage form such as a tablet or capsule. In another embodiment, the compound of formula I is supplied as a powder (e.g., lyophilized powder) that can be resuspended in a liquid suitable for parenteral administration.

Pharmaceutical compositions including an HDACi and tubulin inhibitor, such as eribulin (or pharmaceutically acceptable salts thereof, such as mesylate salt) can be prepared using standard methods known in the art. Pharmaceutical compositions used in the invention can be prepared by, for example, mixing or dissolving the active ingredient(s), having the desired degree of purity, in a physiologically acceptable diluent, carrier, excipient, or stabilizer (see, e.g., Remington's Pharmaceutical Sciences (20th edition), ed. A. Gennaro, 2000, Lippincott, Williams & Wilkins, Philadelphia, Pa.). Acceptable diluents include water and saline, optionally including buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine, or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the formulations of the invention contain a pharmaceutically acceptable preservative. In some embodiments the preservative concentration ranges from 0.1 to 2.0%, typically v/v. Suitable preservatives include those known in the pharmaceutical arts, such as benzyl alcohol, phenol, m-cresol, methylparaben, and propylparaben. Further, the tubulin inhibitor such as eribulin (or pharmaceutically acceptable salts thereof, such as mesylate salts) and/or HDACi formulations optionally include a pharmaceutically acceptable salt, such as sodium chloride at, for example, about physiological concentrations. Thus, in one example, eribulin (or a pharmaceutically acceptable salt thereof, such as eribulin mesylate) is formulated in 0.9% Sodium Chloride Injection (USP).

In some examples, the formulations noted above (and others) can be used for parenteral administration of the drugs. Thus, the drugs can be administered by routes including intravenous, intra-tumoral, peri-tumoral, intra-arterial, intra-dermal, intra-vesical, ophthalmic, intramuscular, intradermal, intraperitoneal, pulmonary, subcutaneous, and transcutaneous routes. Other routes can also be used including, for example, transmucosal, transdermal, inhalation, intravaginal, rectal, and oral administration routes.

The dosage of the tubulin inhibitor and/or HDACi (or pharmaceutically acceptable salts thereof, such as mesylate salt) administered may differ markedly depending on the type of target disease, the choice of delivery method, as well as the age, sex, and weight of the patient, the severity of the symptoms, along with other factors, as can be assessed by those of skill in the art.

The drugs can be administered to a patient prior to, simultaneously or sequentially and in either order (e.g., administration of eribulin (or a pharmaceutically acceptable salt thereof, such as eribulin mesylate) prior to an HDACi, such a compound of Formula I, or vice versa). Many regimens used to administer chemotherapeutic drugs involve, for example, administration of a drug (or drugs) followed by repetition of this treatment after a period (e.g., 1-4 weeks) during which the patient recovers from any adverse side effects of the treatment. Typically, the number of cycles of eribulin (or a pharmaceutically acceptable salt thereof, such as eribulin mesylate) and/or an HDACi, such as a compound of Formula I administration are 4-8, e.g., 4-7, or 6. It may be desirable to use both drugs at each administration or, alternatively, to have some (or all) of the treatments include only one of the drugs.

Thus, for example, the daily dosage of eribulin (or a pharmaceutically acceptable salt thereof, such as eribulin mesylate) may be in the range of, e.g., 0.001 mg/m² to about 100 mg/m² (e.g., in the range of about 0.01 mg/m² to about 50 mg/m², 0.1 to about 5 mg/m², or in the range of about 0.7 mg/m² to about 1.5 mg/m², or in any single amount within these ranges (e.g., 1.4 or 1.1 mg/m²)). The drug may be administered as a single dose once a day, week, month, or year, or more than one dose of the drug may be administered per day, week, month, or year. For example, in one administration protocol, the drug may be administered once daily on days 1 and 8 of a 21-day cycle. In another example, the drug may be administered once daily on days 1, 8, and 15 of a 28-day cycle. The drug can be administered over the course of, e.g., 1 minute to 1 hour (or longer), e.g., over 2 to 5 minutes.

More specifically, in one example, a recommended dose of eribulin mesylate is 1.4 mg/m² administered intravenously over 2 to 5 minutes on days 1 and 8 of a 21-day cycle. A recommended dose of eribulin mesylate in patients with mild hepatic impairment (Child-Pugh A) is 1.1 mg/m² administered intravenously over 2 to 5 minutes on days 1 and 8 of a 21-day cycle, while a recommended dose of eribulin mesylate in patients with moderate hepatic impairment (Child-Pugh B) is 0.7 mg/m² administered intravenously over 2 to 5 minutes on days 1 and 8 of a 21-day cycle. Further, a recommended dose of eribulin mesylate in patients with moderate renal impairment (creatinine clearance of 30-50 mL/min) is 1.1 mg/m² administered intravenously over 2 to 5 minutes on days 1 and 8 of a 21-day cycle. In another example, 1.1 mg/m² eribulin mesylate is administered intravenously over 2 to 5 minutes on days 1, 8, and 15 of a 28-day cycle. HBI-8000 can be given at a dose of about 5, 10, 17.5, 25, 30, 32.5, or 50 mg administered orally twice per week (BIW) or three times (TIW) per week. In some embodiments the dose of HDACi inhibitor can be escalated, e.g., for HBI-8000 a dose escalation of 20 mg, 30 mg, and 40 mg. In some embodiments, the HDACi is used first for a period of time to sensitize (prime) cancer cells to eribulin or a pharmaceutically acceptable salt thereof.

The dosing regimens noted above for eribulin or pharmaceutically acceptable salts thereof and an HDACi of formula I typically start on the same “day 1” and different regimens (e.g., any one of those noted above) for the two drugs can be used together. Thus, for example, both drugs may be administered on days 1 and 8 of a 21-day cycle, both drugs may be administered on days 1, 8, and 15 of a 28-day cycle, etc. Alternatively, one drug (e.g., eribulin, or a pharmaceutically acceptable salt thereof, such as eribulin mesylate) may be administered on days 1 and 8 of a 21-day cycle, while the other drug (e.g., HBI-8000) may be administered on days 0, 3, 7, 10, 14, 17, 21. In a further option, eribulin (or a pharmaceutically acceptable salt thereof, such as eribulin mesylate) is administered intravenously over 2 to 5 minutes on days 1 and 8 of a 21-day cycle, while HBI-8000 is administered twice or three times weekly starting on the same day as eribulin, or a pharmaceutically acceptable salt thereof (e.g., eribulin mesylate) at 4-50 mg.

In addition to tubulin inhibitors such has eribulin or pharmaceutically acceptable salts thereof, such as mesylate salt and an HDACi, the methods of the present disclosure may also include the administration of one or more additional therapeutic agents. Among these agents, immunomodulatory agents (e.g., antibodies or vaccines), chemotherapeutic/antitumor agents, antibacterial agents, anti-emetics, and anti-inflammatory agents are suitable.

The methods of the invention can be used to treat or prevent metastases and/or recurrence in a subject (e.g., a human patient) and/or to decrease tumor size in a primary or secondary tumor. The subject may be diagnosed with cancer, at risk for developing cancer, in treatment for cancer, or in post-therapy recovery from cancer. Further, the treatment may be chemotherapeutic alone, although treatment in combination with a surgical procedure to remove or reduce the size of a tumor, radiation therapy, and/or ablation therapy is also envisioned.

Types of cancers that can be treated according to the present methods include, for example, breast cancer, pancreatic cancer, lung cancer, colon cancer, rectal cancer, colorectal cancer, ovarian cancer, endometrial cancer, skin cancer (e.g., melanoma), prostate cancer, brain cancer, head and neck cancer, liver cancer, kidney cancer, bladder cancer, gastric cancer, gastrointestinal cancer, cancer of the blood (e.g., leukemia), cancer of the lymphatic system, liposarcoma, thyroid cancer, bone cancer (e.g., osteosarcoma), and fibrosarcoma.

In embodiments of the disclosure, the breast cancer treated with the combination is advanced metastatic breast cancer.

The present invention is illustrated by the following examples, which are in no way intended to be limiting of the invention.

### EXAMPLES

### Example 1

In the present example, HBI-8000 was tested as monotherapy at 50 mg/kg, and in combination with eribulin mesylate at 0.3 mg/kg and 1.0 mg/kg. The experiment included a vehicle-treated group, and eribulin at 0.3 mg/kg and 1.0 mg/kg, which served as the control groups for analysis of efficacy.

Two study groups were used; in the first group (Study Group A) tumor growth inhibition and survival were measured. Tumors were measured twice per week until the study was ended on Day 21. Each animal was euthanized when its tumor attained the endpoint tumor volume of 1000 mm³ or on the final day of the study, whichever came first, and the time to endpoint (TTE) for each mouse was calculated. Treatment response was determined from an analysis of percent tumor growth delay (% TGD), defined as the percent increase in the median time to endpoint (TTE) for treated versus control mice; and by log-rank significance of differences in survival among groups and regression responses.

In the second group (Study Group B, tumor-bearing mice were monitored for the development of lung metastases until the number of metastatic nodules reached 30-50 per animal. All animals were euthanized on Day 14, the lungs removed and processed for analysis of metastatic burden.

Mice: Female BALB/c mice (Charles River Laboratories) were seven weeks old, with a body weight (BW) range of 15.6 to 20.2 grams on Day 1 of the study. The animals were fed ad libitum water (reverse osmosis, 1 ppm Cl), and NIH 31 Modified and Irradiated Lab Diet® consisting of 18.0% crude protein, 5.0% crude fat, and 5.0% crude fiber. The mice were housed on irradiated Enrich-O'Cobs™ Laboratory Animal Bedding in static microisolators on a 12-hour light cycle at 20-22° C. (68-72° F.) and 40-60% humidity.

Tumor Cells: 4T1 murine mammary carcinoma cells were maintained in Dulbecco's Modified Eagle Medium (DMEM) supplemented with 10% fetal bovine serum and 2 mM glutamine, 100 units/mL penicillin G sodium, 100 μg/mL streptomycin sulfate, and 25 μg/mL gentamicin. Cell cultures were maintained in tissue culture flasks in a humidified incubator at 37° C., in an atmosphere of 5% CO2 and 95% air.

Tumor Implantation: Cells were harvested during exponential growth, and resuspended in cold DMEM. Each mouse was inoculated subcutaneously in the right flank with 1×106 4T1 cells (0.1 mL of cell suspension). Tumors were calipered in two dimensions to monitor growth as their mean volume approached the desired 100-150 mm³ range. Tumor burden was calculated using the formula:

$$Tumor volume ( {mm}^{3} ) = \frac{{w}^{2} \times l}{2}$$

where w=width and 1=length, in mm, of the tumor. Tumor weight may be estimated with the assumption that 1 mg is equivalent to 1 mm³ of tumor volume. Nine days after tumor implantation, which was designated as Day 1 of the study, animals with individual tumor volumes from 108 to 126 mm³ were sorted into treatment groups. For Study Group A the animals were sorted into 6 treatment groups (Table 1; n=10/group) with group mean tumor volume of 117 mm³. For Study Group B the animals were also sorted into 6 treatment groups (Table 1; n=5/group) with group mean tumor volume of 117 mm³.

TABLE 1
Treatment Groups (Study Groups A and B)

| Group | Treatment | Frequency |
|---|---|---|
| Group 1 | Vehicle (2% CMC: 0.1% Tween 80) | p.o., qd × 21 |
| Group 2 | HBI-8000 at 50 mg/kg | p.o., qd × 21 |
| Group 3 | Eribulin mesylate at 0.3 mg/kg | i.v., qowk × 2 |
| Group 4 | Eribulin mesylate at 1.0 mg/kg | i.v., qowk × 2 |
| Group 5 | HBI-8000 at 50 mg/kg plus | p.o., qd × 21 |
| | Eribulin mesylate at 0.3 mg/kg | i.v., qowk × 2 |
| Group 6 | HBI-8000 at 50 mg/kg plus | p.o., qd × 21 |
| | Eribulin mesylate at 1.0 mg/kg | i.v., qowk × 2 |

Tumor Growth Delay: Tumors were measured using calipers twice per week, and each animal was euthanized when its tumor reached a volume of 1000 mm³ or at the end of the study (D47), whichever came first. Animals that exited the study for tumor volume endpoint were documented as euthanized for tumor progression (TP), with the date of euthanasia. The time to endpoint (TTE) for analysis was calculated for each mouse by the following equation:

$$TTE = \frac{{log}_{10} ( endpoint volume ) - b}{m}$$

where TTE is expressed in days, endpoint volume is expressed in mm³, b is the intercept, and m is the slope of the line obtained by linear regression of a log-transformed tumor growth data set. The data set consisted of the first observation that exceeded the endpoint volume used in analysis and the three consecutive observations that immediately preceded the attainment of this endpoint volume. The calculated TTE is usually less than the TP date, the day on which the animal was euthanized for tumor burden. Animals with tumors that did not reach the endpoint volume were assigned a TTE value equal to the last day of the study. In instances in which the log-transformed calculated TTE preceded the day prior to reaching endpoint or exceeded the day of reaching tumor volume endpoint, a linear interpolation was performed to approximate the TTE. Any animal classified as having died from NTR (non-treatment-related) causes due to accident (NTRa) or due to unknown etiology (NTRu) were excluded from TTE calculations (and all further analyses). Animals classified as TR (treatment-related) deaths or NTRm (non-treatment-related death due to metastasis) were assigned a TTE value equal to the day of death.

Treatment Outcome: Treatment outcome was evaluated from tumor growth delay (TGD), which is defined as the increase in the median time to endpoint (TTE) in a treatment group compared to the control group:

TGD=T−C

expressed in days, or as a percentage of the median TTE of the control group:

$$\% TGD = \frac{T - C}{C} \times 100$$

where T=median TTE for a treatment group, and C=median TTE for the designated control group.

Treatment Efficacy: Treatment efficacy may be determined from the tumor volumes of animals remaining in the study on the last day. The MTV (n) was defined as the median tumor volume on the last day of the study in the number of animals remaining (n) whose tumors had not attained the endpoint volume. Treatment efficacy may also be determined from the incidence and magnitude of regression responses observed during the study. Treatment may cause partial regression (PR) or complete regression (CR) of the tumor in an animal. In a PR response, the tumor volume was 50% or less of its Day 1 volume for three consecutive measurements during the course of the study, and equal to or greater than 13.5 mm³ for one or more of these three measurements. In a CR response, the tumor volume was less than 13.5 mm³ for three consecutive measurements during the course of the study. An animal with a CR response at the termination of a study is additionally classified as a tumor-free survivor (TFS). Animals were monitored for regression responses.

Lung Metastasis: Animals were sacrificed at endpoint using isoflurane anesthesia and necropsies were performed to identify metastases. Total counts were obtained by adding the number of foci counted in the superior, middle, inferior, and post-caval lobes of the right lung to the number of foci counted in the left lung. Percent inhibition was defined as the difference between the number of metastatic foci of the designated control group and the number of metastatic foci of the drug-treated group, expressed as a percentage of the number of metastatic foci of the designated control group:

% Inhibition=[1−(#Foci_{drug-treated}/#Foci_{control})]×100

Statistics: Prism (GraphPad) for Windows 6.07 was used for graphical presentations and statistical analyses. The log-rank test, which evaluates overall survival experience, was used to analyze the significance of the differences between the TTE values of two groups. Logrank analysis includes the data for all animals in a group except those assessed as NTR deaths. Two-tailed statistical analyses were conducted at significance level P=0.05. Group median tumor volumes were plotted as a function of time. When an animal exited the study due to tumor burden, the final tumor volume recorded for the animal was included with the data used to calculate the median volume at subsequent time points. Kaplan-Meier plots show the percentage of animals in each group remaining in the study versus time.

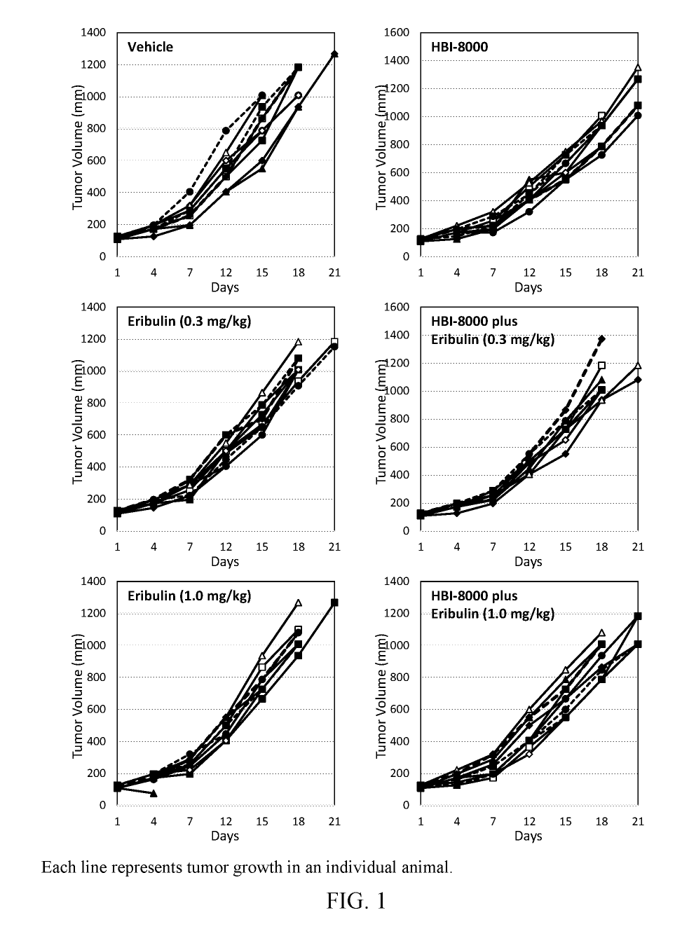

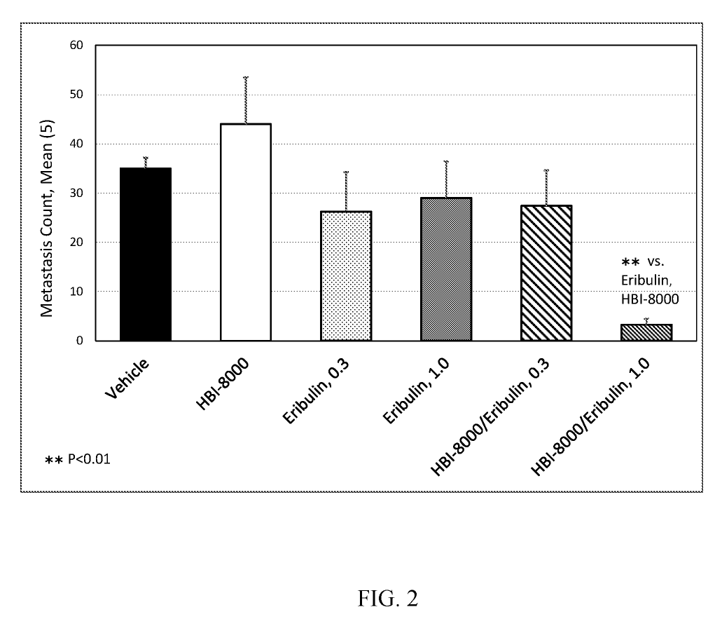

Animals in Example 1 were treated in accordance with the protocol described in Table 1. Table 2 shows the median time to endpoint (TTE) and percent tumor growth delay (% TGD) for all groups in Study Group A. FIG. 1 shows the individual tumor growth curves for all study groups in Study Group A. Tumor incidence and growth were similar in all groups. FIG. 2 depicts the effect of each treatment on the number of metastatic nodules counted for each treatment group in Study Group B. Treatment with the combination of HBI-8000 (50 mg/kg) and eribulin resulted in a statistically significant reduction in metastatic nodules and burden as compared to all other groups, including single agent HBI-8000 or eribulin.

TABLE 2
Median TTE and % TGD for Study Group A (Example 1)

| | | Treatment Regimen | | Median | |
|---|---|---|---|---|---|
| Group | n | Agent 1 | Agent 2 | TTE | % TGD |
| 1 | 10 | vehicle | — | 16.8 | — |
| 2 | 10 | HBI-8000 (50 mg/kg) | — | 18.8 | 12 |
| 3 | 10 | Eribulin mesylate (0.3 mg/kg) | — | 17.8 | 6 |
| 4 | 10 | Eribulin mesylate (1.0 mg/kg) | — | 17.6 | 5 |
| 5 | 10 | HBI-8000 (50 mg/kg) | Eribulin mesylate (0.3 mg/kg) | 17.8 | 6 |
| 6 | 10 | HBI-8000 (50 mg/kg) | Eribulin mesylate (1.0 mg/kg) | 19.4 | 16 |

In summary, none of the single agent or combination therapies had a significant effect on primary tumor growth, though there was a significant ((P<0.01) reduction in the tumor volume distribution after HBI-8000 monotherapy (not shown). Although neither HBI-8000 nor eribulin monotherapies did not significantly inhibit the number of lung metastatic foci in the 4T1 murine mammary carcinoma model in female BALB/c mice, combination therapy with HBI-8000 and eribulin (1 mg/kg dose) produced a foci inhibition of 91%, which was statistically significant (P<0.01) and deemed synergistic.

### Example 2

In the present example, HBI-8000 was tested as monotherapy at 50 mg/kg, and in combination with eribulin mesylate at 1.0 mg/kg. The experiment included a vehicle-treated group, and eribulin at 1.0 mg/kg, which served as the control groups for analysis of efficacy.

Two study groups were used; in the first group, a sentinel group (termed LOOK-SEE) tumor-bearing mice were monitored for the development of lung metastases until the number of metastatic nodules reached 30-50 per animal. After meeting that criteria, all animals in Group B, the main study group, were euthanized on Day 18, the lungs removed and processed for analysis of metastatic burden.

Mice: as described in Example, paragraph [0095]

Tumor Cells: as described in Example, paragraph [0096]

Tumor Implantation: Cells were harvested during exponential growth, and resuspended in cold DMEM. Each mouse was inoculated subcutaneously in the right flank with 0.5×10⁶ 4T1 cells (0.1 mL of cell suspension). Tumors were calipered in two dimensions to monitor growth as their mean volume approached the desired 100-150 mm³ range. Tumor burden was calculated using the formula:

$$Tumor volume ( {mm}^{3} ) = \frac{{w}^{2} \times l}{2}$$

where w=width and 1=length, in mm, of the tumor. Tumor weight may be estimated with the assumption that 1 mg is equivalent to 1 mm3 of tumor volume. Fourteen days after tumor implantation, which was designated as Day 1 of the study, animals with individual tumor volumes from animals with individual tumor volumes ranging from 75 to 108 mm3 were sorted into the “LOOK-SEE” group (n=15) and eight efficacy groups (n=10) with group mean tumor volumes of 95 mm3.

Quantitative PCR (Q-PCR) analysis of tumor gene expression: tumors from all mice were collected on day 18, frozen, and then stored for subsequent analysis. RNA was extracted and tested for quality. For reverse transcription (RT), 2 μg of total RNA was used by using ABI High-Capacity cDNA reverse transcription Kits. For Quantitative PCR 20 μl RT products were diluted with 80 μl nuclease-free H2O to generate 5×-dilution RT products (20 ng/μl). Each PCR reaction included 25 ng cDNA with TaqMan Gene Expression Master Mix and TaqMan Gene Expression Assay. Each sample was tested in triplicate. ViiA™ 7 Real-Time PCR System was used with the following program: (50° C., 2 min), (95° C., 10 min) and 40 cycles (95° C., 15 sec, 60° C., 1 min). Data Analysis was performed using ViiA™ 7 Software v1.2.4 was used for experimental setup and data analysis. Target gene qPCR data were normalized to a reference gene (GAPDH).

Test agents: as described in Example, paragraph [0098].

Treatment: On Day 1 of the study, mice bearing established 4T1 tumors began dosing according to the treatment plan described below. All agents were administered in dosing volumes of 10 mL/kg; volumes were adjusted according to BW of the individual animal.

Group 1 received vehicle, p.o., once daily for sixteen days (qd×16).
Group 2 received HBI-8000 at 50 mg/kg, p.o., qd×16.
Group 3 received eribulin at 1 mg/kg, i.v., qd×1, beginning on D1.
Group 4 received HBI-8000 at 50 mg/kg, p.o., qd×16, and eribulin at 1 mg/kg, i.v., qowk×2.

Treatment efficacy: as described in Example 1, paragraph [0101]

Statistics: as described in Example 1, paragraph [0103]

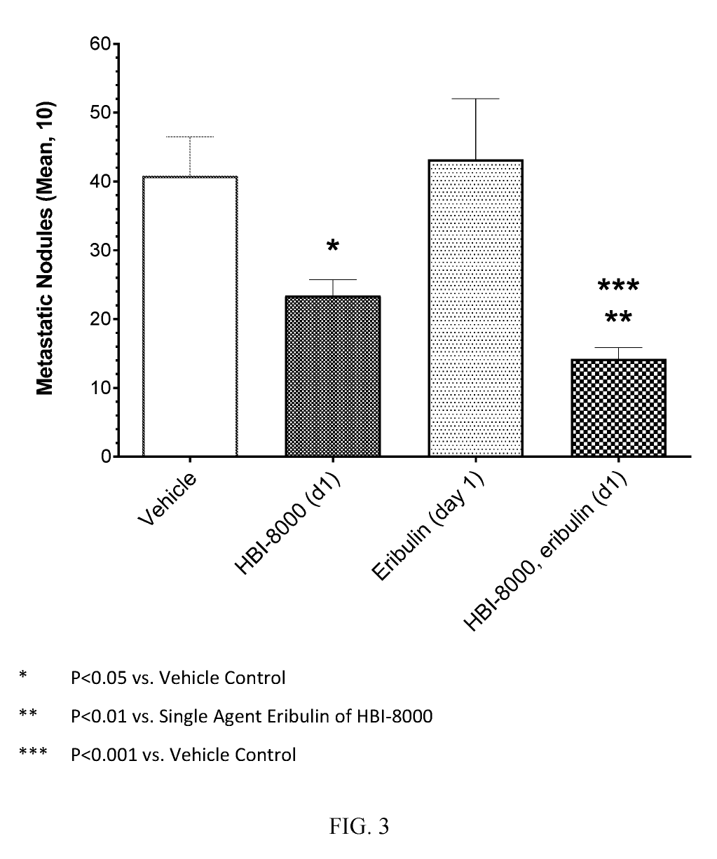

Results: HBI-8000 monotherapy administered on day 1 produced a statistically significant (P<0.05) decrease in metastatic lung foci in 4T1-tumor bearing mice (FIG. 3). Single agent eribulin administered on day 1 did not produce a statistically significant decrease in metastatic lung foci. HBI-8000 plus eribulin combination therapy did produce a decrease in metastatic lung foci which was statistically significant from vehicle control (P<0.005), as well as single agent eribulin (P<0.01) and single agent HBI-8000 (P<0.01).

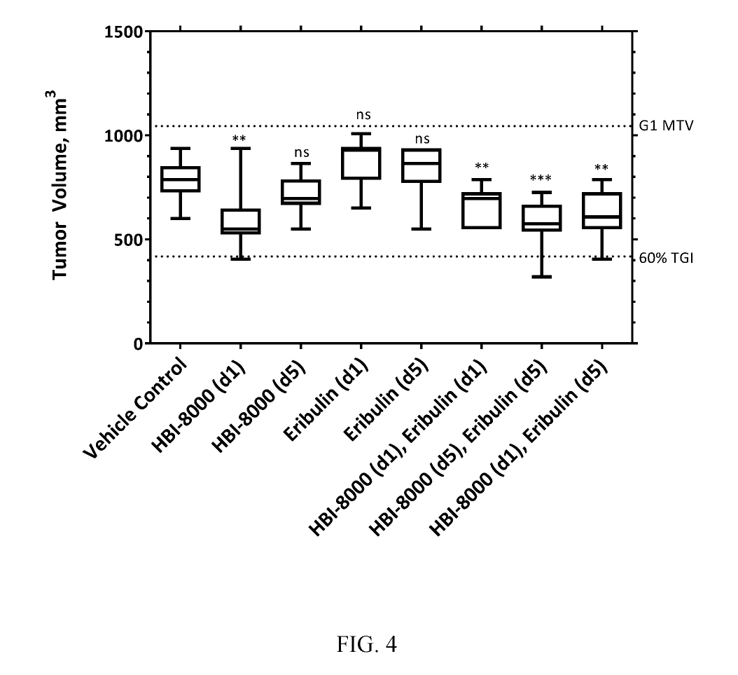

HBI-8000 monotherapy administered on day 1 produced a statistically significant (P<0.001) decrease in primary tumor volume distribution compared to vehicle controls in 4T1-tumor bearing mice (FIG. 4). Single agent eribulin administered on day 1 did not produce a statistically significant decrease in primary tumor volume distribution. HBI-8000 plus eribulin combination therapy did produce a decrease in primary tumor volume distribution which was statistically significant from vehicle control (P<0.001), but not from single agent eribulin or single agent HBI-8000.

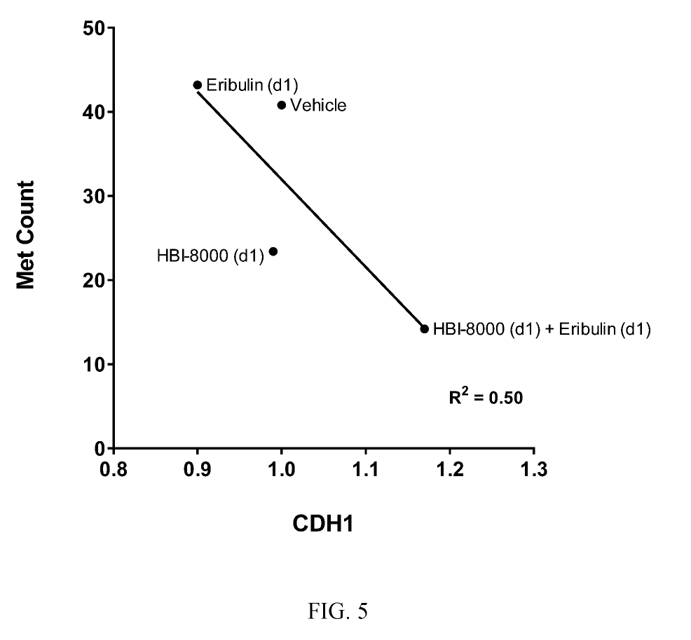

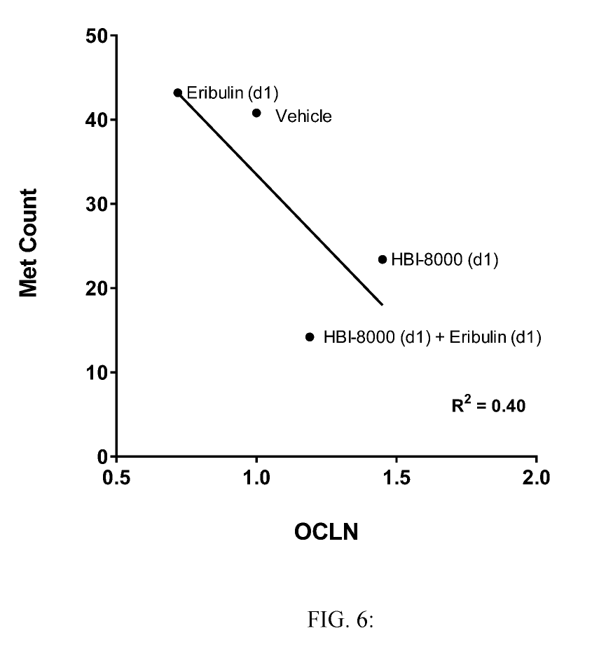

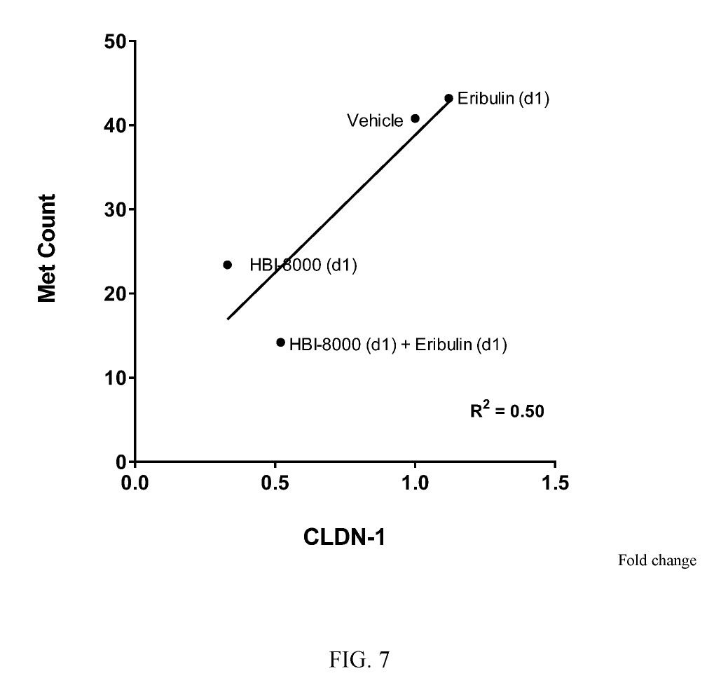

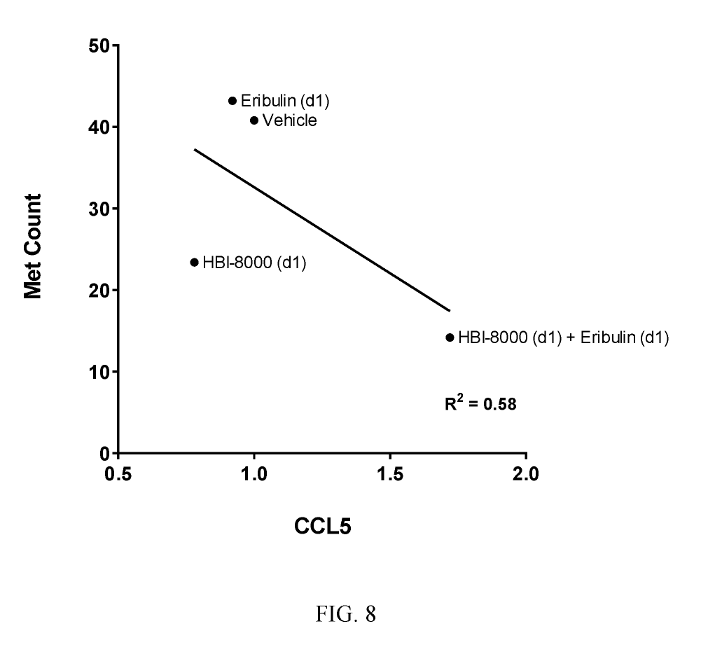

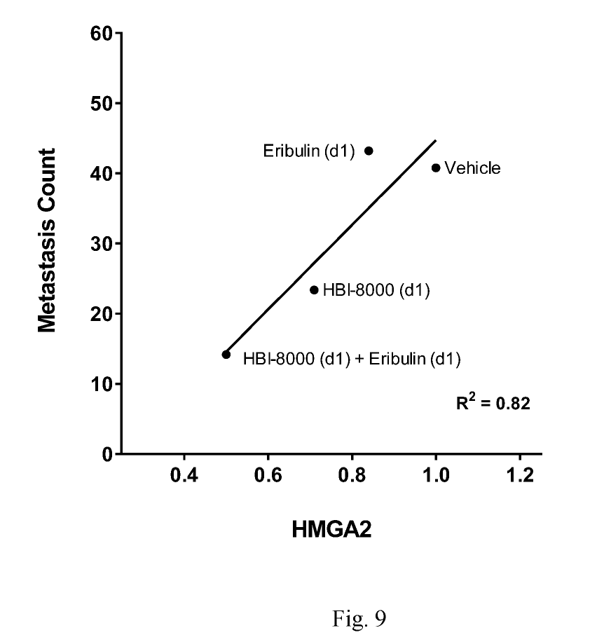

There is accumulating evidence that epithelial-mesenchymal plasticity, referring to the reversible processes of the epithelial-mesenchymal transition (EMT) and the mesenchymal-epithelial transition (MET), plays a role in both treatment resistance and metastatic progression through the acquisition of stemness and invasion programs. Results from the Q-PCR gene expression analysis were analyzed using the linear regression analysis suite in PRISM software to obtain the correlation between the decrease in metastatic lung foci and gene expression for each treatment. The genes analyzed were all known to be important for the process of metastasis and reveal that at least one mechanism of action of HBI-8000 combined with eribulin is the suppression of genes promoting metastasis. These data are shown in FIG. 5-9, and reveal a positive correlation between the increases in CDH1 (e-cadherin) gene and metastasis suppression by HBI-8000 combined with eribulin (FIG. 5). Loss of CDH1 is associated with EMT, tumor invasion and development of metastases. Similarly there were positive correlations between the increase in the OCLN (occludin) (FIG. 6) and CLDN1 (claudin-1) (FIG. 7) genes and metastasis suppression by HBI-8000 combined with eribulin. OCLN is a plasma-membrane protein located at the tight junctions, and is one of the first barriers that must be overcome in the process of breast cancer metastasis. CLDN1 is also a plasma-membrane protein located at the tight junctions, and is also one of the first barriers that must be overcome in the metastatic process of breast cancer. Loss of these genes is associated with EMT, tumor invasion and development of metastases, thus restoring the expression of any one or even all three of these genes can be expected to drive MET and strongly suppress the development of metastasis. There was also a positive correlation between the increases in CCL5 (e-cadherin) gene (FIG. 8) and metastasis suppression by HBI-8000 combined with eribulin. Increased CCL5 expression is associated with Class I HDACi-mediated immunomodulation leading to greater anti-tumor immunity. Finally the strongest correlation between the metastasis suppression by HBI-8000 combined with eribulin was with a decrease in the expression of the HMGA2 (High Mobility Group AT-Hook) gene (FIG. 9). Knockdown of HMGA2, or suppressing HMGA2 expression inhibits EMT plasticity and stemness activities and dramatically reduces tumor growth and metastasis.

Summary: HBI-8000 monotherapy produced a statistically significant decrease in metastatic lung foci in 4T1-tumor bearing mice, however, single agent eribulin did not. HBI-8000 plus eribulin combination therapy did produce a decrease in metastatic lung foci which was statistically significant from vehicle control, as well as single agent eribulin and single agent HBI-8000. Gene expression analysis supports the conclusion that the mechanism for this profound suppression of metastasis is due primarily to the re-expression of genes which drive MET and thus contribute to the inhibition of the development of metastasis, and to the suppression of a key gene known to promote EMT and by extension, promote metastasis.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

1-22. (canceled)

23. A method for treating a breast cancer in a subject, comprising administering to said subject a therapeutically effective amount of a compound of formula I: 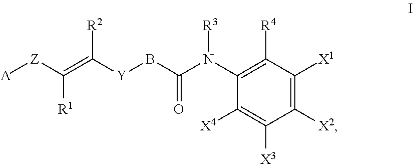
or a pharmaceutically acceptable salt thereof, in combination with a tubulin inhibitor;
wherein:
A is phenyl or a heterocyclic group, optionally substituted with 1 to 4 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkythio, C₁-C₄ perfluoroalkyl, C₁-C₄ perfluoroalkyloxy, C₁-C₄ alkoxycarbonyl, phenyl, and a heterocyclic group;
B is phenyl optionally substituted with 1 to 3 substituents selected from the group consisting of halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, C₁-C₄ alkoxycarbonyl, and phenyl;
Y is —C(O)NH—CH₂—;
Z is a bond or C₁-C₄ alkylene, —O—, —S—, —NH—, —CO—, —CS—, —SO—, or —SO₂—;
R¹ and R² are independently hydrogen or C₁-C₄ alkyl;
R³ is hydrogen or C₁-C₄ alkyl;
R⁴ is hydrogen or —NH₂;
one of X¹, X², X³, or X⁴ is halogen, —OH, —NH₂, —NO₂, —CN, —COOH, C₁-C₄ alkyl, C₁-C₄ alkoxy, C₁-C₄ aminoalkyl, C₁-C₄ alkylamino, C₂-C₄ acyl, C₂-C₄ acylamino, C₁-C₄ alkylthio, C₁-C₄ perfluoroalkyl, C₁-C₄ perfluoroalkyloxy, or C₁-C₄ alkoxycarbonyl optionally substituted with halogen or C₁-C₄ alkyl, while the others of X¹, X², X³, or X⁴ are independently hydrogen,
provided, however, that when R⁴ is hydrogen, one of X¹, X², X³, or X⁴ is —NH₂, a C₁-C₄ aminoalkyl group or a C₁-C₄ alkylamino group.

24. The method of claim 23, wherein the tubulin inhibitor is eribulin, paclitaxel, epothilone, docetaxel, discodermolide, colchicine, combrestatin, 2-methoxyestradiol, methoxy benzenesulfonamides (E7010), vinblastine, vincristine, vinorelbine, vinfluine, dolastatins, halichondrins, hemiasterlins, cryptophysin 52, or a pharmaceutically acceptable salt thereof.

25. The method of claim 23, wherein the breast cancer is metastatic breast cancer.

26. A method for treating a breast cancer in a subject, comprising administering to said subject a therapeutically effective amount of a compound of the following formula: 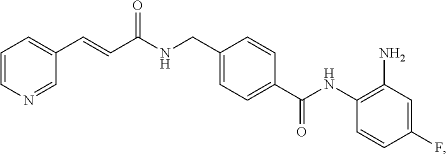 [SMILES: Nc1cc(F)ccc1NC(=O)c1ccc(CNC(=O)/C=C/c2cccnc2)cc1]
or a pharmaceutically acceptable salt thereof, in combination with a tubulin inhibitor.

27. The method of claim 26, wherein the tubulin inhibitor is eribulin, paclitaxel, epothilone, docetaxel, discodermolide, colchicine, combrestatin, 2-methoxyestradiol, methoxy benzenesulfonamides (E7010), vinblastine, vincristine, vinorelbine, vinfluine, dolastatins, halichondrins, hemiasterlins, cryptophysin 52, or a pharmaceutically acceptable salt thereof.

28. The method of claim 27, wherein the tubulin inhibitor is eribulin, or a pharmaceutically acceptable salt thereof.

29. The method of claim 28, wherein the tubulin inhibitor is eribulin mesylate.

30. The method of claim 29, wherein eribulin mesylate is administered intravenously.

31. The method of claim 30, wherein eribulin mesylate is administered intravenously at a dose of 1.4 mg/m² over 2 to 5 minutes on days 1 and 8 of a 21-day cycle.

32. The method of claim 26, wherein the compound is administered orally.

33. The method of claim 32, wherein the compound is administered orally at a dose of about 5, 10, 17.5, 25, 30, 32.5, or 50 mg twice per week (BIW) or three times per week (TIW).

34. The method of claim 26, wherein the breast cancer is metastatic breast cancer.

35. The method of claim 26, wherein the breast cancer is triple negative breast cancer.

36. The method of claim 26, wherein said treatment results in one or more of the following: (i) reduction in the number of cancer cells; (ii) reduction in tumor volume; (iii) increase in tumor regression rate; (iv) reduction in or slowing of cancer cell infiltration into peripheral organs; (v) reduction in or slowing of tumor metastasis; (vi) reduction in or inhibition of tumor growth; (vii) prevention or delay of occurrence and/or recurrence of the cancer, and/or extending of disease- or tumor-free survival time; (viii) increase in overall survival time; (ix) reduction in the frequency of treatment; (x) reduction in cancer burden, and (XI) relieving of one or more of symptoms associated with the cancer.

37. The method of claim 26, wherein said treatment results in reduction in tumor metastasis.

38. The method of claim 37, wherein metastasis to one of more of the adrenal gland, brain, spinal cord, bone, lung, liver, pleura, gastrointestinal tract, peritoneum, muscle, lymph nodes and skin is reduced.

39. The method of claim 37, wherein metastasis to lung is reduced.

40. The method of claim 26, wherein before administering the compound in combination with the tubulin inhibitor, the compound is administered as a single agent.

41. The method of claim 26, further comprising treating the subject with an E-selectin inhibitor, or plerixafor, or a combination of an E-selectin inhibitor and plerixafor.
